(19) United States
(12) Patent Application Publication
(10) Pub. No.: US 2008/0305189 A1
(43) Pub. Date: Dec. 11, 2008

(54) TREATMENT OF ASPIRIN RESISTANCE WITH RADIX SALVIAE MILTIORRHIZAE, ITS EXTRACT AND COMPOSITION

(75) Inventors: Xijun YAN, Tianjin (CN); Naifeng WU, Tianjin (CN); Zhengliang YE, Tianjin (CN); Xu LI, Tianjin (CN); Zhixin GUO, Tianjin (CN); Na ZHAO, Tianjin (CN)

Correspondence Address: HARNESS, DICKEY & PIERCE, P.L.C., P.O. BOX 828, BLOOMFIELD HILLS, MI, 48303, US

(73) Assignee: Tianjin Tasly Pharmaceutical Co., Ltd., Tianjin (CN)

(21) Appl. No.: 12/194,879

(22) Filed: Aug. 20, 2008

Related U.S. Application Data
(62) Division of application No. 10/564,661, filed on Sep. 19, 2006.

(30) Foreign Application Priority Data
Aug. 28, 2003 (CN) 030155275.7
Jun. 30, 2004 (CN) 200410019838.1

(51) Int. Cl. A61K 36/25; A61P 9/00

(52) U.S. Cl. 424/728

(57) ABSTRACT

The present invention relates to the treatment of aspirin resistant cardio-cerebrovascular diseases using RSM (RSM), its extract and composition comprising any or both of them, especially the formulation Dan Shen Drop Pills (Drop Pills of RSM).

### CROSS-REFERENCE TO RELATED APPLICATIONS

This application is a divisional of U.S. patent application Ser. No. 10/564,661 filed Sep. 19, 2006, which is a National Phase filing of PCT/CN2004/000989 filed on Aug. 26, 2004. This application claims the benefit of Chinese Patent Application No. 03155275.7 filed Aug. 28, 2003 and Chinese Patent Application No. 200410019838.1 filed Jun. 30, 2004. The disclosures of the above applications are incorporated herein by reference.

### TECHNICAL FIELD

The present invention relates to medicine. In particular, the present invention relates to the treatment of aspirin resistant cardio-cerebrovascular diseases using Radix Salviae Miltiorrhizae (RSM), its extract and composition comprising any or both of them, especially the formulation Danshen prop Pills (Drop Pills of RSM).

### BACKGROUND

Aspirin belongs to a class of medicines known as non-steroidal anti-inflammatory drugs (NSAIDs). It is an effective anti-inflammatory drug with both analgesic and antipyretic effects. It works by blocking the production of prostaglandin. The most commonly known side effects of aspirin include:
Gastrointestinal reactions, such as poor appetite, peptic ulcers, and in some cases, even perforation
Allergic reaction
Acute renal failure and chronic interstitial nephritis, etc.

As researches become more and more extensive, aspirin's indications have been enlarged from the treatment of fever, relieving mild to moderate pain of various kinds such as headache, toothache, neuralgia, arthralgia, myalgia and dysmenorrhea, and the treatment of rheumatism to the treatment and secondary prevention of cerebral arteriosclerosis, coronary heart disease, and myocardial infarction.

At present, aspirin is widely used in the treatment of cardiovascular disease. The mechanism is that aspirin can block the production of thromboxane A2 (TXA2) in vivo. As TXA2 can promote platelet conglutination and coagulation, aspirin can reduce the incidences of arteriosclerosis and myocardial infarction by inhibiting platelet aggregation. But researches have found that the biosynthesis of thromboxane A2 is not effectively prevented in some patients after they take the drug. That is, aspirin looses its protective effects on cerebrovascular and cardiovascular systems. This is called aspirin resistance (AR). For most patients, aspirin can reduce the risk of cardiovascular disease by 25%, but for patients with aspirin resistance, administration of aspirin, instead of protecting from cardiovascular events, can increase the incidence of myocardial infarction and stroke. These findings have limited the application of aspirin.

At present, there are not so many reports on the treatment of aspirin resistance. Yusuf S etc. reported that, in patients with acute coronary syndrome receiving aspirin, including those undergoing percutaneous coronary intervention, administration of clopidogrel in addition to treatment with aspirin is beneficial in reducing the incidence of early and long-term major cardiovascular events. [“Effects of pretreatment with clopidogrel and aspirin followed by long-term therapy in patients undergoing percutaneous coronary intervention: the PCI-CURE study”. Lancet, 2001].

At present, for researches on aspirin resistance, immunoenzyme assay is commonly used for the measurement of the level of urinary 11-dehydrothromboxane B₂ (TXB₂) in the urine sample of patients before taking drug. 11-dehydrothromboxane B₂ is a metabolite of thromboxane A₂. High urinary 11-dehydrothromboxane B₂ level can identify patients with aspirin resistance and drugs having effects on easing aspirin resistance. It is based on this foundation that this invention is completed.

The Chinese traditional medicine for treating blood disorders, especially for activating blood circulation to dissipate blood stasis, is the commonly used medicine by doctors of all ages. This kind of medicine has many effects, such as promoting blood flow to regulate menstruation, removing blood stasis to eliminate disease, and promoting the subsidence of swelling and the regeneration of tissue. Modern pharmacological studies have confirmed that drugs used to treat blood disorders have many actions such as dilating coronary artery, increasing the coronary blood flow, reducing the oxygen consumption of cardiac muscle, reducing peripheral vascular resistance, inhibiting platelet aggregation, improving microcirculation, inhibiting thrombosis, increasing the activities of fibrinolysis, regulating anticoagulative system, lowering blood pressure, and relieving smooth muscle spasm, etc. Rhizoma Chuanxiong, RSM, Herba Leonuri, Semen Persicae, Flos Carthami, and Hirudo are among drugs used to treat blood disorders. Their clinical applications are developing continuously, and particularly the studies of RSM and its preparations are outstanding.

RSM comes from salvia, a perennial herb of salvia family. It is bitter in taste, and slightly cold in nature. It has efficacies of activating blood circulation to dissipate blood stasis, enriching blood and easing mind, cooling blood and expelling carbuncle, and expelling toxin and promoting tissue regeneration. It is a drug commonly used in traditional Chinese medicine for activating blood circulation to dissipate blood stasis. The main ingredients of RSM are liposoluble diterpenes, and water-soluble phenolic acids. In addition it contains flavonoids, triterpenes and sterols, etc. Among its diterpenes ingredients, tanshinone □, □A, □_{R}, V and □, cryptotanshinone, isotanshinone I, II and IIB, dihydrotanshinone I and so on have a quinoid or ketoform structure. Water soluble ingredients of phenolic acids include danshensu, protocatechualdehyde, protocatechuic acid, caffeic acid, and derivatives of danshensu and caffeic acid, or depsides that are formed by esterification of dimers, such as salvianolic acid A, B, C, D, E and G, alkannic acid B, rosemary acid, methyl ester of rosemary acid, etc. Tanshinone IIA is one of the active ingredients of diterpenes for activating blood circulation to dissipate blood stasis. RSM is currently an important Chinese herbal medicine in the treatment of cardiovascular disease.

Modern researches have confirmed that RSM has pharmacological actions on:
coronary artery
myocardium repair and regeneration
microcirculation
hemorheology
blood lipids.
   These are manifested in dilating the coronary artery, anti-myocardial ischemia, anti-clotting, anti-thrombosis, sedation, alleviating pain, anti-atherosclerosis, and reducing blood lipids, etc.

The major effect of RSM on blood is the inhibition of platelet aggregation induced by adenosine diphosphate (ADP). For patients with blood stasis, the dense and sticky blood condition causes slow blood flow and the platelets tend to adhere to the endangium that has been damaged. Drugs used for activating blood circulation and dissipating blood stasis can improve hemorheology and reduce the adhesion and aggregation of platelets. Furthermore, these drugs can reduce the surface activity of platelets.

The mechanism of action of inhibition of platelet aggregation by drugs used in activating blood circulation to dissipate blood stasis is as follows: platelet aggregation is closely related to the metabolism and activity of prostaglandin and cyclic nucleotide system. Platelet thromboxane (TXA₂) is biosynthesized from phospholipids through many steps and using arachidonic acid as its intermediate. This process is essentially catalyzed by phosphatidase A and cyclooxygenase. The activities of these enzymes are regulated by cAMP which inhibits the activities of these enzymes and therefore the biosynthesis of TXA₂. If cAMP is reduced, the biosynthesis of TXA₂ increases. TXA₂ can promote the release of Ca from sarcoplasmic reticulum which is a reservoir of calcium in platelets. Ca acts on the dense granules causing adenosine diphosphate (ADP) and 5-hydroxytryptamine (5-HT) to release from them. As ADP and 5-HT are the potent promoters for platelet aggregation, the concentration of cAMP is the key factor in platelet aggregation. Increased cAMP reduces platelet aggregation. Furthermore, RSM has the activity of increasing fibrinolysis through activating the profibrinolysin-fibrinolysin system. Also, RSM can shorten the time of serum prothrombin formation.

Radix Notoginseng belongs to a class of traditional Chinese medicine used for hemostasia. In traditional Chinese medicine, it is regarded as having actions of dissipating blood stasis and arresting bleeding, and eliminating swelling and alleviating pain. It has the action of arresting bleeding as well as activating blood. Modern pharmacological researches have confirmed that Radix Notoginseng has both the hemostasis action and anti-clotting action. The hemostatic action includes slowing the bleeding and the hemoglutination progress, increasing platelets quantity and promoting the occurrence of pseudopod stretching, aggregation, and degranulation, etc. It can also reduce the permeability of blood capillary. Ingredients contained in Radix Notoginseng that have anti-clotting action include Radix Notoginseng sponin, Notoginsenoside diol and triol. They all inhibit platelet aggregation in human and rabbit. Radix Notoginseng sponin also promotes the secretion of tissue type profibrinolysin from the blood endothelial cells, and prevents the formation of thrombus.

Borneolum is a crystal product obtained by processing dammar, a resin obtained from plants of the dipteroarpaceae family. The crystal obtained from leaves of Blumea balsamifera Dc, a plant of the Composite family, by steam distillation, is called Praeparatio Blumeae Folii. Products synthesized and processed from camphor, terebenthene and so on through chemical methods are called Borneolum Syntheticum. Borneolum is pungent and bitter in nature. As it is aromatic, it can have a strong stimulating action on the sense organs, and dispel the stagnated fire. Its effects of inducing and promoting resuscitation are similar to Pingxiang (a Chinese medicine). The main ingredient of Borneolum is d-borneol. Praeparatio Blumeae Folii's main ingredient is l-borneol. Modern pharmacological researches have confirmed that Borneolum has effects against myocardial ischemia, and can significantly increase coronary blood flow. Furthermore, as Borneolum can increase the permeability of blood-brain barrier, it can allow more drugs to cross the barrier.

In recent years, through clinical observations and experimental studies, the application of RSM preparations, particularly Compound Danshen Drop Pills (CDDPs, compound RSM drop pills), has further been broadened. Now it has been confirmed that it has effects against angina pectoris, and the effects of improving myocardial ischemia, reducing blood viscosity and platelet aggregation and so on. As a preparation for treatment and prevention of coronary heart disease and angina pectoris, the effect of CDDPs on the hemorheology has been confirmed. Huang Weilan etc. have carried out a comparison study on normal mice and stress stimulated mice, and found that the hemorheological indexes of the mice of both groups were improved significantly after they were given CDDPs. For example, platelet aggregation rate in 1 min and maximum aggregation rate were reduced (P<0.05), and content of plasmatic fibrin was reduced (P<0.01). These indicated a weakening of the thrombosis characteristics. In stress stimulated mice the shear rate was reduced significantly from blood viscosity under the condition of η1˜100 (P<0.05), indicating a process of reducing of blood circulation resistance and strengthening of circulation. Furthermore, there were observed reduction in filtration rate and enhancement in deformability of erythrocyte, but the packed cell volume was unchanged. These indicate that it is upon improving the quality of red blood cell that CDDPs achieve reduction of blood viscosity and improvement of circulation. In mice that did not undergo stimulation, although the whole blood viscosity was reduced, the reduction was insignificant (P>0.05). This means that the improvement of hemorheology by CDDPs in the abnormal hemorheological condition is more significant than in the normal hemorheological condition. Recent researches have found that CDDPs have effects of anti-atherosclerosis, lowering blood lipid, anti-fibrosis of chronic hepatopathy and so on. But up till now, there is not any reliable report about the actions of RSM preparations, particularly CDDPs, on aspirin resistance.

### SUMMARY OF THE INVENTION

One aspect of the present invention relates to the use of RSM and its extract in the treatment of aspirin resistant cardiovascular and cerebrovascular diseases.

Or the purpose of the present invention is to provide the use of RSM and its extract in the preparation of a medicament for treating aspirin resistance.

Said aspirin resistance refers to an inability to effectively inhibit the biosynthesis of thromboxane A₂ after taking aspirin. That is, aspirin loses its protective effect on cardiovascular and cerebrovascular system. In the majority of patients, aspirin can reduce the risk of cardiovascular and cerebrovascular diseases by 25%. But for patients with aspirin resistance, treatment of cardiovascular and cerebrovascular diseases with aspirin can not prevent them from the cardiovascular and cerebrovascular events, but instead, can increase the risk of myocardial infarction and stroke. These findings have restricted the application of aspirin. In the present invention these cardiovascular and cerebrovascular diseases are called aspirin resistant cardiovascular and cerebrovascular diseases, particularly coronary heart disease and angina pectoris in which aspirin treatment is ineffective. In this invention, drugs that are effective in the treatment of aspirin resistant cardiovascular and cerebrovascular diseases are called drugs of anti-aspirin resistance; this action is called effect of anti-aspirin resistance.

The present invention adopts a now commonly used method for research of aspirin resistance. It uses immunoenzyme assay to test the urinary sample of the patients and analyze the change in the level of urinary 11-dehydrothromboxane B₂ (TXB₂) to determine if there is any reduction of aspirin resistance in the patients after taking RSM preparation. From clinical investigations, the present invention confirms that RSM and its extract have effects of reducing aspirin resistance, and can be used as drugs of anti-aspirin resistance and for the preparation of drugs of anti-aspirin resistance.

Another aspect of the present invention relates to the use of a composition that contains RSM as its active ingredient in the treatment of aspirin resistant cardiovascular and cerebrovascular diseases. The compositions of the present invention include compound RSM preparations, particularly CDDPs and compound Danshen tablets (CDT, compound RSM tablets).

The following RSM compositions of this invention have preferable anti-aspirin resistance effect: RSM 30-180 parts, Radix Notoginseng 5-40 parts, Borneolum 0.3-2.5 parts, and adjuvants 10-40 parts. A preferred composition comprises RSM 75-115 parts, Radix Notoginseng 14-20 parts, Borneolum 0.8-1.2 parts, and adjuvants 15-30 parts. The most preferred composition comprises RSM 90 parts, Radix Notoginseng 17.6 parts, Borneolum 1 parts, and adjuvants 20 parts.

The adjuvants used in the compositions of the present invention can be any adjuvants commonly used in the art of pharmaceutics, preferably polyethylene glycol, and most preferably polyethylene glycol 6000.

RSM, its extract and compositions containing them as the active ingredients can be formulated into any pharmaceutical preparation. The preferred preparations are drop pills, spray solution, pellets, pills, granules, capsules, tablets, powder and oral liquid, etc.

For the preparation of active ingredients of the present invention, the following methods can be adopted: water extraction, water extraction combined with ethanol precipitation, extraction, impregnation, percolation, refluxing extraction, consecutive refluxing extraction, and macroporous resin adsorption. For example, these medicinal materials can be ground into powder and are mixed uniformly to form a powder preparation that can be infused orally. These medicinal materials can also be decocted with water, and then concentrated to form an oral liquid. But in order to exert the best effect of every active ingredient of the medicinal materials, it is preferable to use the following process for extraction.

RSM, 90 g; Radix Notoginseng, 17.6 g; Borneolum, 1 g; polyethylene glycol 6000, 20 g are provided. RSM and Radix Notoginseng are decocted with water for three times. The decoctions are pooled and filtrated. The filtrate is condensed. 2 volumes of 95% ethanol are added and the solution is allowed to stand still for 24 hours before filtrating. After the ethanol is recovered from the solution, the solution is condensed to reach a relative density of 1.33˜1.35 (55˜60° C.). Borneolum is dissolved in ethanol of appropriate volume. The above-mentioned solutions of extraction and of borneolum are added into polyethylene glycol that is melted in a water bath and stirred thoroughly. The resulted solution is kept at a temperature of 70±2° C. The solution is dropped by using a dropper of appropriate diameter at a speed of 60-80 drops per minute into liquid paraffin that is cooled in an ice bath. After being shaped, the pills are taken out and mopped out the liquid paraffin from its surface with absorbent paper. 1000 pills are prepared.

Amounts of the above components can be increased or reduced proportionally when in production factory use. If it is in a large scale production, the amounts in kilograms or tons as the measure unit may be used. If it is in a small-scale production, the amounts in grams may be used. The weights of every component may be increased or reduced, but the proportions of the medicinal materials are unchanged.

The said RSM, its extract and compositions comprising them include simple recipe or compound preparations that contain RSM or its extract. These compound preparations can contain other traditional Chinese medicine or chemical drug component.

### DETAILED DESCRIPTION OF THE INVENTION

The present invention is elaborated in details hereinafter. The specific examples or embodiments should not be construed to limit the scope of the application in any way.

### Preparation Examples

### Example 1

### Method for Preparing Danhong Injection (Injection of RSM and Flos Carthami)

1. RSM 750 g, Flos Carthami 250 g and sodium chloride for injection 7 g were prepared for ready use.

2. The RSM was immersed in a diluted warm ethanol for one hour before filtrating. The extraction by immersing was repeated. The filtrates were pooled for use. The residue of RSM was combined with Flos Carthami before immersing in warm water for one hour followed by filtrating. The extraction by immersing was repeated. All the filtrates were pooled and condensed to a clear paste with a relative density of 1.10˜1.20 (65□). Sodium chloride for injection was added into the paste to an isotonic concentration. After the pH was adjusted to 6-7, the paste was filtrated followed by refrigerating for 24 hours. A sufficient quantity of water was added to produce the desired volume. The resulted slurry was filtrated, vialed and sterilized to produce danhong injection.

### Example 2

### Method for Preparing Qianglinaoxinkang Capsules

1. RSM 1500 g, Armillaria Mellea extract 1500 g and royal jelly 125 g were prepared for ready use.

2. The RSM 75 g was crushed into fine powder and the rest RSM was crushed to coarse powder. Water was added into the resulted powder to decoct three times, with the first time and the second time 2 hours and the third time 3 hours. The decoctions were combined and filtrated. The filtrate was condensed under decompression condition to a thick paste with a relative density of 1.30˜1.32 (70□). The paste was evaporated under decompression condition and crushed into fine powder. Armillaria Mellea extract was condensed under decompression condition to a thick paste having a relative density of 1.30˜1.32 (70□). The paste was evaporated under decompression condition and crushed into fine powder. The fine powder was combined with the above-mentioned fine powder and mixed thoroughly. The mixture was granulated and desiccated. The royal jelly was freeze-dried to obtain fine powder. The powder was combined with the above granules and mixed thoroughly before encapsulating to produce the titled capsules.

### Example 3

### Method for Preparing Tongxinshu Capsules

1. Seabuckthorn flavone 250 g, RSM extract 20 g, Rhizoma Chuanxiong 10 g and talcum powder 20 g were prepared for ready use.

2. The Rhizoma Chuanxiong was crushed into fine powder and admixed with seabuckthorn flavone and RSM extract. The talcum powder was added followed by encapsulation to produce the tongxinshu capsules.

### Example 4

### Method for Preparing Compound Dangshen Tablets

1. Radix Codonopsis 704 g, RSM 192 g, Radix Angelicae Sinensis 192 g, Radix Glehniae 128 g, Radix Tinosporae 64 g, starch 4.7 g, dextrin 9.4 g, talcum powder 4.3 g and magnesium stearate 1.6 g were prepared for ready use.

2. The Radix Tinosporae was immersed in water for 2 hours. The rest four medicinal materials including RSM were immersed in water for 1 hour. The extract thus obtained was pooled and decocted three times, each for one hour. The decoctions for three times were pooled and filtrated, followed by condensing and drying under decompression condition at a temperature less than 85□. The dry extract resulted was crushed to coarse powder before adding starch and dextrin. The mixture was mixed up thoroughly and granulated before drying at 85□. The granules were trimmed and the talcum powder and magnesium stearate were added before mixing thoroughly. The mixture obtained was tableted and sugar-coated to produce the desired tablets.

### Example 5

### Method for Preparing Danxiangguanxin Injection

1. RSM 1000 g and Lignum Dalbergiae Odoriferae 100 g were prepared for ready use.

2. The Lignum Dalbergiae Odoriferae was soaked with water and a sufficient quantity of water was added before distilling. About 700 ml distillate was collected and refrigerated for 24 hours. The oil layer was removed and filtrated. The aqueous layer was collected in another container. The RSM was decocted with water for three times, 2 hours for each time. The decoctions were pooled and filtrated before condensing to 500 ml. The ethanol was added to obtain an ethanol content of 75% and the resulted solution was refrigerated for 48 hours before filtrating. The filtrate was condensed to 200 ml by recovering the ethanol. Ethanol was added again to obtain an ethanol content of 85%. The resulted solution was refrigerated for 48 hours before filtrating. The filtrate was condensed to 120 ml by recovering the ethanol. A sufficient quantity of water for injection was added to the desired volume of 1000 ml and refrigerated for 16 hours before filtrating. The filtrate was condensed to a volume of 250 ml and refrigerated for 72 hours. The pH was adjusted to 6.0-6.8 with 10% solution of sodium hydroxide. The active carbon was added in an amount of 0.1%-0.4% of the medicinal materials and boiled for 30 min before filtrating. The filtrate was adjusted to pH 4 with weak hydrochloric acid. The active carbon was added again in an amount of 0.1%-0.4% of the medicinal materials before boiling 30 min. The resulted solution was refrigerated for at least 24 hours before filtrating. The filtrate was adjusted to pH 6.5-7.0 with 10% solution of sodium hydroxide. After the above distillate of Lignum Dalbergiae Odoriferae was added, a sufficient quantity of water for injection was added to the desired volume. The solution was filtrated, vialed and sterilized to obtain the titled injection.

### Example 6

### Method for Preparing Danshen injection

1. RSM 64 g and glucose 50 g (67 g) were prepared for ready use.

2. RSM was decocted with water for three times, 2 hours for each time. The decoctions were pooled and filtrated. The filtrate was condensed to a clear paste with a relative density of 1.16 (70□). Ethanol was added to obtain an ethanol content of 75%. The solution was agitated thoroughly before refrigerating for 24 hours followed by filtrating. The filtrate was condensed to a clear paste with a relative density of 1.06˜1.08 (78□) by recovering the ethanol. The pH was adjusted to 9 with a solution of 40% sodium hydroxide. The paste was boiled for one hour before adjusting the pH to 6 with hydrochloric acid followed by filtrating. After the filtrate was cooled to room temperature, the ethanol was added to obtain an ethanol content of 85%. The solution was agitated thoroughly before refrigerating for 24 hours followed by filtrating. The filtrate was condensed to a clear paste with a relative density of 1.11˜1.13 (78□) by recovering the ethanol. Water for injection was added to dilute the paste 4-folds. After the pH was adjusted to 3 with hydrochloric acid, the diluted paste was refrigerated for 72 hours before filtrating. After boiling the filtrate, active carbon was added in an amount of 0.1% (g/ml). The filtrate was boiled for 15 min. before filtrating. The filtrate was held for future use. The glucose was added to the boiling water for injection to obtain a thick solution of 50%˜60%. Hydrochloric acid q.s was added and active carbon was added in an amount of 0.1% (g/ml) at the same time. The solution was agitated thoroughly, and boiled for 15 min. The solution was filtrated when it was hot to remove the active carbon. The filtrate was combined with the filtrate of RSM. The pH was adjusted to 3.8˜4.2 with a solution of 10% sodium hydroxide. After boiling, active carbon was added in an amount of 0.05% (g/ml) followed by boiling for 30 min. After the solution was filtrated, a sufficient quantity of water for injection was added to the desired volume of 500 ml. Sodium bisulfite 0.5 g was added and mixed thoroughly. The pH was adjusted to 5˜6 with the solution of 10% sodium hydroxide. Water for injection was added to the desired volume before filtrating, fine filtrating and ultrafiltrating. The filtrate was vialed and sterilized to produce the titled injection.

### Example 7

### Method for Preparing Jingzhi Guanxin Granules

1. RSM 350.8 g, Radix Paeoniae Rubra 175.4 g, Rhizoma Chuanxiong 175.4 g, Flos Carthami 175.4 g, Lignum Dalbergiae Odoriferae 116.9 g, sucrose 841 g and dextrin 105 g were prepared for use.

2. The four medicine materials except Flos Carthami were decocted with water for three times, with the first time 2 hours, the second time 1.5 hours, and the last 1 hour before filtrating. The filtrates were pooled and held for future use. Immerse Flos Carthami was immersed in a suitable amount of warm water of 80□ for two times, with the first time 2 hours, and the second time 1 hour before filtrating. The filtrate was combined with the above filtrate and condensed to thick paste. The paste was dried at 80□, then crushed to fine powder. The sucrose and dextrin were added and agitated thoroughly before granulating and drying to obtain the desired granulates.

### Example 8

### Method for Preparing Jingzhi Guanxin Tablets

1. RSM 375 g, Radix Paeoniae Rubra 187.5 g, Rhizoma Chuanxiong 187.5 g, Flos Carthami 187.5 g, Lignum Dalbergiae Odoriferae 187.5 g, starch 12 g and magnesium stearate 5 g were prepared for ready use.

2. Lignum Dalbergiae Odoriferae was distilled to obtain volatile oil and the aqueous solution after distillation was collected in another container. The other four medicine materials including RSM were refluxed two times with 85% ethanol under heating, with the first time 3 hours and the second time 2 hours followed by filtrating. The filtrates were pooled and the ethanol was recovered. The filtrate was combined with the aqueous solution mentioned above and condensed under decompression condition to a thick paste with a relative density of 1.35-1.40 (50□). After the starch was added, the paste was granulated by using 5# starch slurry as binder. After the granulate was dried, the volatile oil of Lignum Dalbergiae Odoriferae was added and agitated thoroughly. The magnesium stearate was added followed by tabletting and sugar-coating or film-coating to produce the titled tablets.

### Example 9

### Method for Preparing Shuxintong Capsules

1. RSM 180 g, Herba Portulacae 180 g, Rhizoma Homalomenae 180 g, Rhizoma Chuanxiong 180 g, Lignum Dalbergiae Odoriferae 200 g and Borneolum 80 g were prepared for ready use.

2. Rhizoma Chuanxiong 60 g, RSM 60 g, Lignum Dalbergiae Odoriferae, and Borneolum were crushed into fine powder respectively and held for future use. The rest Rhizoma Chuanxiong, RSM, Herba Portulacae and Rhizoma Homalomenae were decocted with water for two times, with the first time 1.5 hours and the second time one hour. The decoctions were filtrated and the filtrates were pooled before condensing to a clear paste with a relative density of 1.10-1.20 (90□). Ethanol was added to obtain an ethanol content of 60% and the mixture was held for 48 hours before filtrating. The ethanol was recovered from the filtrate to obtain a thick paste with a relative density of 1.31 (80□). The above-mentioned fine powders were added and mixed thoroughly. The mixture was dried up under at a temperature less than 80□, and crushed into powders. The powders were mixed thoroughly and encapsulated to obtain the titled granules.

### Example 10

### Method for Preparing Xinnaoning Capsules

1. Folium Ginkgo 400 g, Buxus microphylla 400 g, Migao 400 g, RSM 400 g and Bulbus Allii Macrostemi 400 g were prepared for ready use.

2. Bulbus Allii Macrostemi was crushed to fine powder and sieved for use. Migao was distilled with steam and the volatile oil was recovered. The residue of Migao was combined with Folium Ginkgo, Buxus microphylla, RSM and extracted with 75% ethanol. The extract was condensed under decompression condition to a clear paste by removing the ethanol. The paste was held for future use. Water was added into the residue and the mixture was decocted for 1 hour before filtrating. The filtrate was condensed to a clear paste with a relative density of 1.20 (80□). The paste was combined with the paste mentioned above and condensed to a thick paste. Fine powder of Bulbus Allii Macrostemi and the volatile oil were added into the paste and mixed thoroughly before encapsulating to produce the titled capsulate.

### Example 11

### Method for Preparing Danshen Mixture

1. RSM 550 g and simple syrup (65 wt. % of sucrose in water) 265 ml were prepared for ready use.

2. RSM was decocted with water for two times, the first time 3 hours and the second time 2 hours. The decoctions were pooled before filtrating. The filtrate was condensed to 500 ml. The ethanol was added to obtain an ethanol content of 80% and stirred thoroughly before held still for 48 hours. Supernatant was separated and the ethanol was recovered. The solution was condensed to a clear paste having a relative density of 1.12 (60-65□). The paste was diluted with water followed by agitating thoroughly. The resulted solution was refrigerated for 48 hours before filtrating. After the simple syrup was added into the filtrate, water was added to obtain the desired volume followed by agitating thoroughly. The solution was vialed and sterilized to produce the titled mixture.

### Example 12

### Method for Preparing Guanxin Danshao Tablets

1. RSM 650 g, Radix Paeoniae Rubra 325 g, Rhizoma Chuanxiong 325 g, Flos Carthami 325 g, Lignum Dalbergiae Odoriferae 250 g and Radix Acanthopanacis Senticosi 250 g were prepared for ready use.

2. 30 g Radix Paeoniae Rubra was crushed into fine powder for ready use. Lignum Dalbergiae Odoriferae was distilled to obtain volatile oil and the aqueous solution after distillation was collected in another container. The residue of Lignum Dalbergiae Odoriferae and 295 g Radix Paeoniae Rubra and RSM were decocted with water for two times, the first time 3 hours and the second time 2 hours. The decoctions were pooled and filtrated. The filtrate was held for future use. Flos Carthami was immersed in warm water (70-80□) for two times, the first time 3 hours and the second time 2 hours, followed by filtrating. The filtrates were pooled and held for future use. Rhizoma Chuanxiong was extracted by refluxing using 70% ethanol for two times, the first time 8 hours and the second time 6 hours. The extracts were pooled and filtrated. The filtrate was processed by recovering the ethanol. The filtrate of Rhizoma Chuanxiong was combined with the above-mentioned filtrates before condensing to a thick paste with a relative density of 1.30 (80□). The above-mentioned powder was added before drying under decompression condition. The product was crushed to fine powder and sieved. The powder was mix thoroughly before granulating. After the granulates were dried, the volatile oil of Lignum Dalbergiae Odoriferae was sprayed over before mixing thoroughly. The mixture was tableted and film-coated to produce the titled tablets.

### Example 13

### Method for Preparing Xinxinshu Capsules

1. Radix Astragali 600 g, Radix Rehmanniae 360 g, Fructus Schisandrae 180 g, RSM 180 g, Radix Paeoniae Rubra 360 g, Ramulus Cinnamomi 180 g and General Ginsenoside in extract of Ginseng stems and leaves 10 g were prepared for ready use.

2. One third of the recipe amount of Radix Paeoniae Rubra was crushed into fine powder for ready use. The rest of Radix Paeoniae Rubra together with RSM were extracted by refluxing using ethanol for two times, each 2 hours. The extracts were pooled and filtrated. Filtrate was condensed to a thick paste with a relative density of 1.36 (60□) by recovering the ethanol. The paste was vacuum-desiccated and crushed into fine powder for ready use. The residue of the ethanol extraction and the rest four medicines including Radix Astragali were decocted with water for three times, the first time 2 hours, the second time 1.5 hours and the third time 1 hour. The decoctions were pooled and filtrated. The filtrate was condensed to a thick paste of relative density 1.36 (60□) under decompression condition. The fine powder of Radix Paeoniae Rubra was added and mixed thoroughly. The mixture was vacuum-desiccated and crushed into fine powder for ready use. The General Ginsenoside in extract of Ginseng stems and leaves were combined with the above two fine powders and mixed thoroughly. The mixture was granulated by using a suitable amount of ethanol followed by drying and encapsulating to obtain the titled capsules.

### Example 14

### Method for Preparing Danshen Tablets

1. RSM 1000 g and starch 210 g were prepared for ready use.

2. RSM was extracted by refluxing using 90% ethanol for 1.5 hours. The extract was filtrated and ethanol was removed from the filtrate. Water was added to the residue before decocting for one hour. The decoction was filtrated and condensed to the appropriate volume before combining with the extract by ethanol. The extract was further condensed to a thick paste with a relative density of 1.30 (90□). The starch was added and mixed thoroughly. The mixture was desiccated, granulated, tableted and sugar-coated to obtain the titled tablets.

### Example 15

### Method for Preparing Xiongxiang Tongmai Pills

1. Rhizoma Chuanxiong 30 g, Fructus Chebulae 20 g, RSM 30 g, Semen Myristicae 15 g, Styrax 1.5 g, Borneolum 0.75 g, Moschus 0.15 g g and polyethylene glycol 6000 15 g were prepared for ready use.

2. RSM, Rhizoma Chuanxiong, Fructus Chebulae, and Semen Myristicae were crushed into coarse powders and extracted by using supercritical CO₂ respectively. The extracts were pooled and polyethylene glycol 6000 was added. Then Borneolum, Styrax and the above fine powder were added successively and mixed thoroughly. The mixture was held at 80˜85□ and was dropped into pills by using methyl silicone oil as the cooling agent. Thus the titled pills were produced.

### Example 16

### Method for Preparing Compound Danshen Capsules

1. RSM 450 g, Radix Notoginseng 141 g, Borneolum 8 g and Beta cyclodextrin 40 g were prepared for ready use.

2. Radix Notoginseng was crushed into fine powder for future use. Borneolum was dissolved in a suitable amount of ethanol. Water was added into Beta cyclodextrin and the mixture was kept in a thermostatic water bath of 55□ to resolve Beta cyclodextrin by agitation. The mixture was stirred continuously in water bath for 30 minutes and ethanol solution of Borneolum was dropped at the same time. Then the water bath was removed and the solution was refrigerated and pump-filtrated. The residue after pump-filtration was baked at 40□ and held for future use. RSM was extracted for three times as follows. At the first time, RSM was extracted by refluxing for 1.5 hours using ethanol. The extract was filtrated and the filtrate was condensed to a thick paste with a relative density of 1.30 (55˜60□). At the second time, it was extracted by refluxing for 1.5 hours using 50% ethanol and the extract was filtrated. At the third time, it was extracted by refluxing for 2 hours using water and the extract was filtrated. The filtrates obtained at the second time and the third time were pooled and condensed to a thick paste with a relative density of 1.40 (55˜60□). The paste obtained at the first time was added and stirred thoroughly to produce a thick paste having a relative density of 1.35-1.39 (55˜60□). The fine powder of Radix Notoginseng was added and mixed thoroughly. The obtained mixture was desiccated and crushed into fine powder. Beta cyclodextrin mixture was added and mixed thoroughly before encapsulating to produce the titled capsules.

### Example 17

### Method for Preparing Compound Danshen Drop Pills

1. RSM 90.0 g, Radix Notoginseng 17.6 g and Borneolum 1.0 g were prepared for ready use.

2. RSM and Radix Notoginseng were crushed into powders and water was added in an amount of 6-folds of the medicinal materials to extract the medicinal materials for three times at a temperature between 80˜90□, the first time 3 hours, the second time 2 hours, and the third time one hour. The extracts were filtrated and the filtrates were pooled before condensing under decompression condition. Ethanol was added into the condensed filtrate to obtain an ethanol content of 55-71% so that sedimentation was resulted in. The supernatant was recovered and ethanol was removed from the supernatant before condensing to obtain a thick paste with a relative density of 1.20˜1.25 (50˜65□). Borneolum and polyethylene glycol 6000 as much as 7-folds of the paste were added. The mixture was dropped into the liquid paraffin of 2˜8□ at the temperature of 85˜95□ to produce 1000 pills.

### Example 18

### Method for Preparing Compound Danshen Tablets (CDT)

1. RSM 450 g, Radix Notoginseng 141 g and Borneolum 8 g were prepared for ready use.

2. RSM was extracted for three times as follows. At the first time, RSM was extracted by refluxing for 1.5 hours using ethanol. The extract was filtrated and ethanol was removed from the filtrate before condensing the filtrate into a thick paste with a relative density of 1.30 (55˜60□). At the second time, it was extracted by refluxing for 1.5 hours using 50% ethanol and the extract was filtrated. At the third time, it was extracted by refluxing for 2 hours using water and the extract was filtrated. The filtrates obtained at the second time and the third time were pooled and ethanol was recovered from the filtrate. The filtrate was condensed to a thick paste with a relative density of 1.40 (55˜60□). The paste obtained at the first time was added and stirred thoroughly to produce a clear paste having a relative density of 1.35-1.39 (55˜60□). Radix Notoginseng was crushed into fine powders. The powders were added into the paste and mixed thoroughly. The obtained mixture was desiccated and granulated. Borneolum was crushed into fine powder and the powder was combined with the granules and mixed thoroughly. The mixture was tableted to produce 1000 tablets, which was sugar-coated or film-coated to obtain the titled tablets.

### Example 19

### Method for Preparing Guanxin Danshen Tablets

1. RSM 200 g, Radix Notoginseng 200 g and oil of Lignum Dalbergiae Odoriferae 1.75 ml were prepared for ready use.

2. Radix Notoginseng was crushed into fine powder. RSM was crushed into medium powder and the powders were percolated using 90% ethanol as the solvent. The percolate was condensed to thick paste. The residue of the percolated material was decocted with water for two times, each one hour. The decoctions were pooled and filtrated before condensing to the desired volume. The above fine powder and thick paste were added into the condensed filtrate and stirred thoroughly. The mixture was granulated and desiccated. The oil of Lignum Dalbergiae Odoriferae was sprayed over the granules and mixed thoroughly. The mixture was tableted to produce 1000 tablets before sugar-coating to obtain the titled tablets.

### Example 20

### Method for Preparing Guanxin Danshen Drop Pills

1. RSM 200 g, Radix Notoginseng 200 g and oil of Lignum Dalbergiae Odoriferae 1.75 ml were prepared for ready use.

2. Radix Notoginseng was crushed into fine powder. RSM was crushed into medium powder and the powders were percolated using 90% ethanol as the solvent. The percolate was condensed to thick paste. The residue of the percolated material was decocted with water for two times, each one hour. The decoctions were pooled and filtrated before condensing to the desired volume. The above fine powder and thick paste were added into the condensed filtrate and stirred thoroughly. An appropriate amount of polyethylene glycol was added and the oil of Lignum Dalbergiae Odoriferae was sprayed over before mixing thoroughly. The mixture was melted and dropped into a liquid paraffin of 0˜10□ to produce 1000 the titled drop pills.

### Example 21

(a) RSM 45 g, Radix Notoginseng 8 g, Borneolum 0.5 g, xylitol 12 g and xanthan gum 3 g were prepared for ready use.

(b) RSM and Radix Notoginseng were decocted with water for 5 times. The decoctions were pooled and filtrated. The filtrate was condensed before 90% ethanol in an amount of 2-folds of the condensed filtrate was added. The solution was held still for 20 hours before filtrating. Ethanol was recovered from the filtrate. The filtrate was condensed to a thick paste (1) with a relative density of 1.15˜1.25 (50□).

(c) Borneolum was dissolved in an appropriate amount of ethanol to obtain solution (2). Paste (1) and solution (2) were added into a mixture of xylitol and starch melted in a water bath. The mixture was stirred thoroughly and kept warmly. The mixture was dropped into methyl silicone oil at a temperature between 55˜75□ and at a speed of 30˜60 drops/min. to make the pills with a dropper having the caliber of 1.30˜4.0 mm. 1000 pills were produced. After the pills were shaped, they were taken out and mopped on surface with absorbent paper. Thus, the desired pills were obtained.

### Example 22

(a) RSM 150 g, Radix Notoginseng 20 g, Borneolum 1.5 g, lactitol 83 g and starch 17 g were prepared for ready use.

(b) RSM and Radix Notoginseng were decocted with water for 2 times. The decoctions were pooled and filtrated. The filtrate was condensed before 85-95% ethanol in an amount of 4-folds of the condensed filtrate was added. The solution was held still for 36 hours before filtrating. Ethanol was recovered from the filtrate. The filtrate was condensed to a clear paste (1) with a relative density of 1.10˜1.25 (70-80□).

(c) Borneolum was dissolved in an appropriate amount of ethanol to obtain solution (2). Paste (1) and solution (2) were added into a mixture of lactitol and starch melted in a water bath. The mixture was stirred thoroughly and kept warmly. The mixture was dropped into plant oil at a temperature between 65˜95□ and at a speed of 20˜50 drops/min. to make the pills with a dropper having the caliber of 1.10˜3.0 mm. 1000 pills were produced. After the pills were shaped, they were taken out and mopped on surface with absorbent paper. Thus, the desired pills were obtained.

### Example 23

(a) RSM 100 g, Radix Notoginseng 15 g, Borneolum 0.8 g, xylitol 37.5 g and arabic gum 12.5 g were prepared for ready use.

(b) RSM and Radix Notoginseng were decocted with water for 3 times. The decoctions were pooled and filtrated. The filtrate was condensed before 85-90% ethanol in an amount of 4-folds of the condensed filtrate was added. The solution was held still for 18 hours before filtrating. Ethanol was recovered from the filtrate. The filtrate was condensed to a thick paste (1) with a relative density of 1.05˜1.15 (40-50□).

(c) Borneolum was dissolved in an appropriate amount of ethanol to obtain solution (2). Paste (1) and solution (2) were added into a mixture of xylitol and arabic gum. The mixture was stirred thoroughly for 10 to 30 minutes and kept warmly to be melted at temperature between 50˜95□. The mixture was dropped into methyl silicone oil at a temperature between 60˜85□ and at a speed of 50˜60 drops/min. to make the pills with a dropper having the caliber of 1.1˜3.5 mm. 1000 pills were produced. After the pills were shaped, they were taken out and mopped on surface with absorbent paper. Thus, the desired pills were obtained.

### Example 24

(a) RSM 75 g, Radix Notoginseng 14 g, Borneolum 1.2 g, xylitol 30.7 g and arabic gum 8.3 g were prepared for ready use.

(b) RSM and Radix Notoginseng were decocted with water for 3 times. The decoctions were pooled and filtrated. The filtrate was condensed before 95% ethanol in an amount of 3-folds of the condensed filtrate was added. The solution was held still for 24 hours before filtrating. Ethanol was recovered from the filtrate. The filtrate was condensed to a thick paste (1) with a relative density of 1.20˜1.30 (50-75□).

(c) Borneolum was dissolved in an appropriate amount of ethanol to obtain solution (2). Paste (1) and solution (2) were added into a mixture of xylitol and arabic gum and stirred thoroughly. The mixture was melted at a temperature between 50˜115□ and stirred thoroughly for 10 to 30 minutes. The mixture was kept warmly and dropped at a temperature between 60˜85□ and at a speed of 20˜60 drops/min. into liquid paraffin at a temperature between 0˜18□ to make the pills with a dropper having the caliber of 1.1˜3.5 mm. 1000 pills were produced. After the pills were shaped, they were taken out and mopped on surface with absorbent paper. Thus, the desired pills were obtained.

### Example 25

(a) RSM 115 g, Radix Notoginseng 20 g, Borneolum 1.0 g, xylitol 36 g and pregelatine starch 4 g were prepared for ready use.

(b) RSM and Radix Notoginseng were decocted with water for 2-4 times. The decoctions were pooled and filtrated. The filtrate was condensed before 90-97% ethanol in an amount of 1 to 3-folds of the condensed filtrate was added. The solution was held still for 18-30 hours before filtrating. Ethanol was recovered from the filtrate. The filtrate was condensed to a thick paste (1) with a relative density of 1.20˜1.40 (50-75□).

(c) Borneolum was dissolved in an appropriate amount of ethanol to obtain solution (2). Paste (1) and solution (2) were added into a mixture of xylitol and pregelatine starch melted in water bath and stirred thoroughly. The mixture was melted at a temperature between 80˜95□ and stirred thoroughly for 10 to 30 minutes. The mixture was kept warmly and dropped at a temperature between 60˜65□ and at a speed of 40˜60 drops/min into methyl silicone at a temperature of 0˜18□ to make the pills with a dropper having the caliber of 1.2˜2.5 mm. 1000 pills were produced. After the pills were dried, they were packaged. Thus, the desired pills were obtained.

### Example 26

(a) RSM 40 g, Radix Notoginseng 17.6 g, Borneolum 1 g, sorbitol 15.5 g and starch 4.5 g were prepared for ready use.

(b) RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a thick paste (1) with a relative density of 1.20˜1.40 (60□).

(c) Borneolum was dissolved in an appropriate amount of ethanol to obtain solution (2). Paste (1) and solution (2) were added into a mixture of sorbitol and starch and stirred thoroughly. The mixture was melted at a temperature between 50˜75□ and stirred thoroughly for 10 to 30 minutes. The mixture was kept warmly and dropped at a temperature between 60˜65□ and at a speed of 60˜80 drops/min. into liquid paraffin at a temperature between 0˜18□ to make the pills with a dropper having the caliber of 1.1˜3.5 mm. 1000 pills were produced. After the pills were shaped, they were taken out and mopped on surface with absorbent paper. Thus, the desired pills were obtained.

### Example 27

(a) RSM 90 g, Radix Notoginseng 17.6 g, Borneolum 1 g, xylitol 14.6 g and Carrageenan 5.4 g were prepared for ready use.

(b) RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a thick paste (1) with a relative density of 1.33˜1.35 (55˜60□).

(c) Borneolum was dissolved in an appropriate amount of ethanol to obtain solution (2). Paste (1) and solution (2) were added into a mixture of xylitol and carrageenan and stirred thoroughly. The mixture was melted at a temperature between 80˜115□ and stirred thoroughly for 10 to 30 minutes. The mixture was kept warmly and dropped at a temperature of 70±2□ and at a speed of 60˜80 drops/min. into liquid plant oil at a temperature between 0˜18□ to make the pills with a dropper having the caliber of 1.1˜3.5 mm. 1000 pills were produced. After the pills were shaped, they were taken out and mopped on surface with absorbent paper. Thus, the desired pills were obtained.

### Example 28

(a) RSM 90 g, Radix Notoginseng 17.6 g, Borneolum 1 g, lactitol 16 g and starch 4 g were prepared for ready use.

(b) RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a thick paste (1) with a relative density of 1.33˜1.35 (55˜60□).

(c) Borneolum was dissolved in an appropriate amount of ethanol to obtain solution (2). Paste (1) and solution (2) were added into a mixture of lactitol and starch and stirred thoroughly. The mixture was melted at a temperature of 64□ and stirred thoroughly for 10 to 30 minutes. The mixture was kept warmly and dropped at a temperature of 64□ and at a speed of 20˜60 drops/min. into methyl silicone oil at a temperature of 0□ to make the pills with a dropper having the caliber of 1.2˜2.5 mm. 1000 pills were produced. After the pills were shaped, they were taken out and mopped on surface with absorbent paper. After the pills were dried, they were packaged to obtain the desired pills.

### Example 29

(a) RSM 90 g, Radix Notoginseng 17.6 g, Borneolum 1 g, xylitol 14 g and arabic gum 6 g were prepared for ready use.

(b) RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a thick paste (1) with a relative density of 1.33˜1.35 (55˜60□).

(c) Borneolum was dissolved in an appropriate amount of ethanol to obtain solution (2). Paste (1) and solution (2) were added into a mixture of xylitol and arabic gum and stirred thoroughly. The mixture was melted at a temperature of 64□ and stirred thoroughly for 10 to 30 minutes. The mixture was kept warmly and dropped at a temperature of 64□ and at a speed of 20˜40 drops/min. into methyl silicone oil at a temperature of 10□ to make the pills with a dropper having the caliber of 1.2˜2.5 mm. 1000 pills were produced. After the pills were shaped, they were taken out and mopped on surface with absorbent paper. After the pills were dried, they were packaged to obtain the desired pills.

### Example 30

(a) RSM 41.06 g, Radix Notoginseng 8.03 g, Borneolum 0.46 g, xylitol 12 g and arabic gum 8 g were prepared for ready use.

(b) RSM and Radix Notoginseng were crushed and decocted in an extraction pot together with 5 volumes water for 2 hours. The decoction was filtrated. The residue was decocted together with 4 volumes water for one hour to extract again. The decoction was filtrated. The filtrates were pooled and condensed under decompression condition to obtain a ratio of 1:0.9˜1.1 between the volume of the condensed solution (L) and the weight of medicine materials (Kg). 95% ethanol was added slowly to obtain an ethanol content of 69˜71%. The solution was held still for 12 hours to carry on ethanol precipitation. Supernatant was collected and filtrated. Ethanol was recovered from the filtrate. The filtrate was condensed to a thick paste (1) with a relative density of 1.32˜1.40.

(c) The paste and Borneolum were added into a mixture of xylitol and arabic gum and stirred thoroughly. The mixture was melted at a temperature of 64□ and stirred thoroughly for 10 to 30 minutes. The mixture was kept warmly and dropped at a temperature of 64□ into methyl silicone oil at a temperature of 0□ to make the pills with a dropper having the caliber of 1.2˜2.5 mm. The pills were taken out and mopped on surface with absorbent paper. After the pills were dried, they were packaged to obtain the desired pills.

### Example 31

(a) RSM 59.36 g, Radix Notoginseng 6.38 g, Borneolum 0.34 g, lactitol 11 g and arabic gum 9 g were prepared for ready use.

(b) RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a thick paste (1) with a relative density of 1.33˜1.35 (55˜60□).

(c) Borneolum was dissolved in an appropriate amount of ethanol to obtain solution (2). Paste (1) and solution (2) were added into a mixture of lactitol and arabic gum. The mixture was melted at a temperature of 75□ and stirred thoroughly for 10 to 30 minutes. The mixture was kept warmly and dropped at a temperature of 70□ and at a speed of 30˜80 drops/min. into methyl silicone oil at a temperature of 0□ to make the pills with a dropper having the caliber of 1.2˜2.5 mm. 1000 pills were produced. The pills were taken out and mopped on surface with absorbent paper. After the pills were dried, they were packaged to obtain the desired pills.

### Example 32

(a) RSM 41.06 g, Radix Notoginseng 8.03 g, Borneolum 0.46 g, lactitol 15 g, carboxymethyl starch 3 g and arabic gum 2 g were prepared for ready use.

(b) RSM and Radix Notoginseng were crushed and decocted in an extraction pot together with 5 volumes water for 2 hours. The decoction was filtrated. The residue was decocted together with 4 volumes water for one hour to extract again. The decoction was filtrated. The filtrates were pooled and condensed under decompression condition to obtain a ratio of 1:0.9˜1.1 between the volume of the condensed solution (L) and the weight of medicine materials (Kg). 95% ethanol was added slowly to obtain an ethanol content of 69˜71%. The solution was held still for 12 hours to carry on ethanol precipitation. Supernatant was collected and filtrated. Ethanol was recovered from the filtrate. The filtrate was condensed to a thick paste (1) with a relative density of 1.32˜1.40.

(c) The paste and Borneolum were added into a mixture of lactitol, carboxymethyl starch and arabic gum in a weight ratio of 1:0.2˜1:0.4 and stirred thoroughly. The mixture was melted at a temperature of 80˜95□ and stirred thoroughly for 10 to 30 minutes. The mixture was kept warmly and dropped at a temperature of 60˜85□ into plant oil at a temperature of 0˜18□ to make the pills with a dropper having the caliber of 1.21˜2.5 mm. The pills were taken out, dried, and packaged to obtain the desired pills.

### Example 33

(a) The paste 7 g as prepared in Example 28, Borneolum 0.1 g, xylitol 18.5 g and starch 1.5 g were prepared for ready use.

(b) Xylitol and starch were mixed thoroughly and added into the mixture of paste and Borneolum. The mixture was melted at a temperature of 75□ and stirred thoroughly for 10 to 30 minutes. The mixture was kept warmly and dropped at a temperature of 60˜70□ and at a speed of 50˜60 drops/min. into methyl silicone oil at a temperature of 0□ to make the pills with a dropper having the caliber of 1.2˜2.5 mm. 1000 pills were produced. After the pills were shaped, they were taken out and mopped on surface with absorbent paper. The pills were dried and packaged to obtain the desired pills.

### Example 34

(a) The RSM and Radix Notoginseng paste 12.5 g as prepared in Example 21, Borneolum 3.2 g, lactitol 20 g and arabic gum 3.5 g were prepared for ready use.

(b) Lactitol and arabic gum were mixed thoroughly and melted at a temperature of 55˜85□. The above-mentioned paste and Borneolum were added and stirred thoroughly for 10 to 30 minutes. The mixture was kept warmly and dropped at a temperature of 60˜75□ and at a speed of 30˜50 drops/min. into plant oil at a temperature of 5˜10□ to make the pills with a dropper having the caliber of 1.2˜2.5 mm. 1000 pills were produced. The pills were taken out and mopped on surface with absorbent paper. After the pills were dried, they were packaged to obtain the desired pills.

### Example 35

(a) The RSM and Radix Notoginseng paste 5.5 g as prepared in Example 27, Borneolum 0.5 g, xylitol 16.5 g and starch 3.5 g were prepared for ready use.

(b) Xylitol and starch were mixed thoroughly and melted at a temperature of 60˜85□. The above-mentioned paste and Borneolum were added and stirred thoroughly for 10 to 30 minutes. The mixture was kept warmly and dropped at a temperature of 60˜85□ and at a speed of 20˜60 drops/min. into liquid paraffin at a temperature of 5˜15□ to make the pills with a dropper having the caliber of 1.2˜2.5 mm. 1000 pills were produced. The pills were taken out and mopped on surface with absorbent paper. After the pills were dried, they were packaged to obtain the desired pills.

### Example 36

(a) The RSM and Radix Notoginseng paste 4.65 g as prepared in Example 24, Borneolum 0.85 g, xylitol 15.5 g and arabic gum 4.5 g were prepared for ready use.

(b) Xylitol and arabic gum were mixed thoroughly and melted at a temperature of 65˜85□. The above-mentioned paste and Borneolum were added and stirred thoroughly for 10 to 20 minutes. The mixture was kept warmly and dropped at a temperature of 60˜65□ and at a speed of 20˜40 drops/min. into plant oil at a temperature of −10˜15□ to make the pills with a dropper having the caliber of 1.21˜2.5 mm. 1000 pills were produced. The pills were taken out and mopped on surface with absorbent paper. After the pills were dried, they were packaged to obtain the desired pills.

### Example 37

### Preparation Method for CDDP

RSM 90 g, Radix Notoginseng 17.6 g, Borneolum 1 g and polyethylene glycol 6000 20 g were prepared for ready use.

RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a paste (1) with a relative density of 1.33˜1.35 (55˜60□).

Borneolum was dissolved in an appropriate amount of ethanol to obtain solution (2). Paste (1) and solution (2) were added into polyethylene glycol melted in water bath and stirred thoroughly. The mixture was kept at a temperature of 70±2□ and dropped at a speed of 60˜80 drops/min. into liquid paraffin cooled in ice bath to make the pills with a dropper having the suitable caliber. 1000 pills were produced. After the pills were shaped, they were taken out and mopped on surface with absorbent paper to obtain the desired pills.

### Example 38

### Preparation Method for CDDP

RSM 180 g, Radix Notoginseng 25 g, Borneolum 2 g and polyethylene glycol 6000 30 g were prepared for ready use.

RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a paste (1) with a relative density of 1.33˜1.35 (55˜60□).

Borneolum was dissolved in an appropriate amount of ethanol to obtain solution (2). Paste (1) and solution (2) were added into polyethylene glycol melted in water bath and stirred thoroughly. The mixture was kept at a temperature of 70±2□ and dropped at a speed of 60˜80 drops/min. into liquid paraffin cooled in ice bath to make the pills with a dropper having the suitable caliber. 1000 pills were produced. After the pills were shaped, they were taken out and mopped on surface with absorbent paper to obtain the desired pills.

### Example 39

### Preparation Method for CDDP

RSM 115 g, Radix Notoginseng 14 g, Borneolum 1.2 g and polyethylene glycol 6000 40 g were prepared for ready use.

RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a paste (1) with a relative density of 1.33˜1.35 (55˜60□).

Borneolum was dissolved in an appropriate amount of ethanol to obtain solution (2). Paste (1) and solution (2) were added into polyethylene glycol melted in water bath and stirred thoroughly. The mixture was kept at a temperature of 70±2□ and dropped at a speed of 60˜80 drops/min. into liquid paraffin cooled in ice bath to make the pills with a dropper having the suitable caliber. 1000 pills were produced. After the pills were shaped, they were taken out and mopped on surface with absorbent paper to obtain the desired pills.

### Example 40

### Preparation Method for CDDP

RSM 30 g, Radix Notoginseng 40 g, Borneolum 0.3 g and polyethylene glycol 6000 40 g were prepared for ready use.

RSM and Radix Notoginseng were decocted with water for 3 times. The decoctions were pooled and filtrated. The filtrate was condensed before 95% ethanol in an amount of 2-folds of the condensed filtrate was added. The solution was held still for 24 hours before filtrating. Ethanol was recovered from the filtrate. The filtrate was condensed to a paste (1) with a relative density of 1.33˜1.35 (55˜60□).

Borneolum was dissolved in an appropriate amount of ethanol to obtain solution (2). Paste (1) and solution (2) were added into polyethylene glycol melted in water bath and stirred thoroughly. The mixture was kept at a temperature of 70±2□ and dropped at a speed of 60˜80 drops/min. into liquid paraffin cooled in ice bath to make the pills with a dropper having the suitable caliber. 1000 pills were produced. After the pills were shaped, they were taken out and mopped on surface with absorbent paper to obtain the desired pills.

### Test Example 1

### Reduction of Aspirin Resistance by Compound Danshen Drop Pills

Between October 2003 and January 2004, a screening survey of aspirin resistance for retired personnel and their couples in over 20 sanatoriums affiliated with Beijing Military Region, and identified 86 cases of aspirin resistance (56 male, 29 female, and the average age of patients: 70.9±10.9). The incidence of aspirin resistance was about 15.6%. We divided the patients with aspirin resistance into two groups: combination therapy group (55 patients, administered aspirin and compound danshen drop pills (CDDP), referred to as “combination therapy group” hereinafter); compound danshen drop pills group (31 patients, only administered compound danshen drop pills, referred to as “CDDP group” hereinafter).

Methods:

1. Enrolment Criteria:
Those who incessantly administered low dose aspirin over two weeks
Those who had the maximum platelet aggregation rate induced by arachidonic acid larger than 30% measured by the American CHRON-LOG AGGREGOMETER 540VS.

Subjects conformed with both of the above conditions were enrolled in the study.

2. Exclusion Criteria:

Those having any one of the following diseases:

a) Blood system disease, particularly bleeding disease

b) Cancer

c) Chronic obstructive pulmonary diseases

Subjects conformed with any one of the above criteria should be excluded.

3. Grouping

Combination therapy group: randomly selected 55 patients with aspirin resistance administered aspirin continuously, and simultaneously administered CDDP for two weeks (30 pills/day).

CDDP group: randomly selected 31 patients with aspirin resistance administered CDDP instead of Aspirin for two weeks (30 pills/day).

4. Effect Evaluation Criteria
remarkably effective: maximum platelet aggregation rate less than 30% (equal to the normal effect of aspirin)
effective: maximum platelet aggregation rate more than 30%, but less than 80% (the rate fell in the normal range of platelet activation rate, but worse than the normal inhibition effect of aspirin)
ineffective: variation of maximum aggregation rate before and after treatment ranged between ±10%
elevated: maximum aggregation rate after the treatment elevated more than 10%

effectiveness=(number of remarkably effective patients+number of effective patients)/number of total patients

Results

1. Combination Therapy Group

(1) Effectiveness of treatment

| Treatment effect | Patients | Ratio % |
|---|---|---|
| remarkably effective | 46 | 83 |
| effective | 1 | 2 |
| ineffective | 1 | 2 |
| elevated | 7 | 13 |

The effectiveness of combination therapy group was up to 85% (47/55).

(2) Comparison of the Maximum Platelet Aggregation Rates Before and after the Treatment:

t—test: Paired samples mean analysis

| | Variable 1 | Variable 2 |
|---|---|---|
| average | 69.45454545 | 27.76363636 |
| variance | 517.8821549 | 714.776431 |
| observed value | 55 | 55 |
| poisson correlation coefficient | −0.06556464 | |
| Supposed average deviation | 0 | |
| df | 54 | |
| t Stat | 8.534597132 | |
| P(T,, t) one tailed | 6.82878E−12 | |
| t one tailed critical | 1.673565748 | |
| P(T,, t) two tailed | 1.36576E−11 | |
| T two tailed critical | 2.004881026 | |

It could be seen from the above table that the platelet aggregation rate before the treatment was 69.5±22.8 and it became 27.8±26.7 after 2 weeks treatment (p<0.01, paired t-test). The mean reduction of platelet aggregation rate was 51.57%.

Note: reduction=(value before treatment−value after treatment)/value before treatment

2. CDDP Group

(1) Effectiveness of treatment

| Treatment effect | Patients | ratio % |
|---|---|---|
| remarkably effective | 12 | 39 |
| effective | 3 | 10 |
| ineffective | 5 | 16 |
| elevated | 11 | 35 |

The effectiveness of CDDP group was up to 49% (15/31)

(2). Comparison of Maximum Platelet Aggregation Rates Before and after the Treatment t-Test: Paired Samples Mean Analysis

t-test: Paired samples mean analysis

| | Variable 1 | Variable 2 |
|---|---|---|
| average | 69.45454545 | 27.76363636 |
| variance | 517.8821549 | 714.776431 |
| observed value | 55 | 55 |
| poisson correlation coefficient | −0.06556464 | |
| supposed average deviation | 0 | |
| df | 54 | |
| t Stat | 8.534597132 | |
| P(T,, t) one tailed | 6.82878E−12 | |
| t one tailed critical | 1.673565748 | |
| P(T,, t) two tailed | 1.36576E−11 | |
| T two tailed critical | 2.004881026 | |

It could be seen from the above table that the platelet aggregation rate before the treatment was 80.4±12.1, and it became 62.8±38.2 after 2 weeks treatment (p<0.05, paired t-test). The mean reduction of platelet aggregation rate was 18.75%.

Note: reduction=(value before treatment−value after treatment)/value before treatment

Conclusion: Combination therapy of aspirin resistance by administering aspirin and CDDP simultaneously could significantly reduce platelet aggregation rate. In combination therapy group, the effectiveness was 85%, and the average reduction was 51.57%. In CDDP group, the effectiveness was 49%, and the average reduction was 18.75%. The results showed that combination therapy by administering aspirin and CDDP simultaneously could treat aspirin resistant patients who could not be treated by administering aspirin alone. Administration of CDDP could also treat aspirin resistant patient, but the effect was not so good as combination therapy. It needed further research to see whether CDDP reduces aspirin resistance by other mechanism of action.

### Test Example 2

### Reduction of Aspirin Resistance by CDDP

This study adopted a reviewing method, and the normal value referred to the research results about aspirin resistance in Australia.

General Information

All 178 patients, including 140 males and 38 females, came from the retired personnel of the First Sanatorium, Bafenbu Sanatorium. The patients were aged 70˜87 and the average age was 77.1. 170 patients were randomly selected for sample test and the number of patients whose data could be accepted was 150 (IC20˜IC80). Of those enrolled in the study, in addition to aspirin resistance, 91 patients were afflicted with hypertension, 25 patients were afflicted with hyperlipidemia, 32 patients were afflicted with diabetes, 139 patients were afflicted with coronary heart disease, and 57 patients were afflicted with cerebrovascular diseases. To treat aspirin resistance, 50 patients administered aspirin (aspirin group), 50 patients administered CDDP (CDDP group), and 50 patients administered both aspirin and CDDP (combination therapy group). All the patients administered drugs for at least half a year before sampling.

Materials and Methods

The urinary sample was the middle section of the first urina of the day. The urinary sample was placed in the liquid nitrogen immediately after sampling and stored at −86□ in a low-temperature refrigerator. The urinary 11-dehydrothromboxane B₂ (TXB₂) was determined by immunoenzyme assay using the reagent kit of Cayman Chemical company. All the experiments were completed in the Biological Institute of Tasly Research Academy. Information about patient grouping was double-blind for researchers and statisticians.

Results

T-test was conducted for average value between groups.

TABLE 1
Values of urinary 11-dehydrothromboxane B₂ in patients of each group
(ng/mmol)

| | | | Combination therapy |
|---|---|---|---|
| | Aspirin group | CDDP group | group |
| | (n = 50) | (n = 50) | (n = 50) |
| | Urinary | Urinary | Urinary |
| | 11-dehydro TXB₂ | 11-dehydro TXB₂ | 11-dehydro TXB₂ |
| | (ng/mmol) | (ng/mmol) | (ng/mmol) |
| | 30.24 | 30.94 | 20.55 |
| | 19.00 | 13.00 | 10.43 |
| | 18.78 | 19.36 | 30.27 |
| | 30.46 | 12.23 | 18.64 |
| | 14.23 | 12.63 | 24.02 |
| | 37.24 | 31.86 | 17.65 |
| | 12.24 | 13.08 | 21.23 |
| | 36.00 | 35.64 | 32.77 |
| | 11.63 | 23.32 | 9.25 |
| | 37.61 | 10.38 | 11.44 |
| | 34.64 | 9.52 | 13.27 |
| | 13.41 | 28.60 | 34.98 |
| | 16.68 | 28.01 | 22.03 |
| | 27.60 | 21.94 | 12.77 |
| | 30.12 | 27.32 | 28.92 |
| | 14.74 | 15.21 | 11.01 |
| | 15.54 | 34.42 | 28.12 |
| | 38.25 | 10.67 | 9.74 |
| | 27.68 | 18.80 | 30.25 |
| | 28.94 | 24.25 | 21.05 |
| | 36.51 | 28.67 | 17.43 |
| | 17.45 | 14.25 | 13.33 |
| | 14.23 | 32.43 | 26.52 |
| | 39.35 | 10.25 | 8.59 |
| | 37.65 | 22.49 | 30.99 |
| | 9.25 | 20.52 | 20.66 |
| | 14.84 | 11.28 | 29.73 |
| | 40.62 | 31.28 | 31.25 |
| | 28.43 | 10.53 | 10.27 |
| | 27.32 | 34.66 | 27.54 |
| | 12.33 | 17.68 | 11.20 |
| | 39.50 | 23.11 | 9.04 |
| | 18.25 | 10.99 | 31.75 |
| | 37.42 | 13.48 | 12.80 |
| | 26.26 | 19.50 | 14.70 |
| | 30.44 | 32.70 | 29.00 |
| | 16.82 | 29.82 | 8.23 |
| | 9.96 | 23.52 | 26.00 |
| | 9.24 | 15.00 | 8.74 |
| | 26.43 | 30.24 | 14.70 |
| | 24.22 | 28.62 | 29.99 |
| | 30.24 | 15.43 | 27.74 |
| | 34.45 | 26.25 | 18.43 |
| | 30.22 | 13.99 | 19.70 |
| | 9.78 | 35.89 | 22.05 |
| | 27.63 | 10.02 | 37.85 |
| | 10.10 | 14.23 | 7.90 |
| | 36.38 | 20.82 | 10.82 |
| | 45.27 | 21.96 | 26.25 |
| | 15.24 | 21.45 | 19.07 |
| X ± SD | 25.02 ± 10.51 | 21.25 ± 8.28 | 20.21 ± 8.61 |

Conclusion: The excreted TXB2 between combination therapy group and aspirin group was significantly different in statistics (t=2.50 p<0.05). The excreted TXB2 between CDDP group and aspirin group was significantly different in statistics (t=1.99 p<0.05). The excreted TXB2 between combination therapy group and CDDP group was not significantly different in statistics (t=0.62 p>0.05).

The experiment data demonstrated that CDDP could reduce the excretion of urinary TXB2. That is to say, it could block the biosynthesis of thromboxane A₂ (TXA₂) in vivo and reduce platelet conglutination and coagulation. Accordingly, it could inhibit platelet aggregation and reduce the incidence of arteriosclerosis and myocardial infarction. The experiment results indicated that CDDP had anti-aspirin resistance effect. CDDP could improve life indexes in aspirin resistant patients with cardiovascular diseases.

### Test Example 3

### Reduction of Aspirin Resistance by Compound Danshen Tablets

This study used the same method as in test example 2.

150 patients in this study were all selected from the cardiovascular outpatients having the tendency of aspirin resistance. All the patients, including 80 males and 70 females, aged between 70˜85, with the average age of 77. To treat aspirin resistance, 50 patients administered aspirin (aspirin group), 50 patients administered Compound Danshen Tablets (CDT group), and 50 patients administered aspirin and CDT simultaneously (combination therapy group). All the patients administered drugs for at least half a year before sampling.

Materials and Methods

The urinary sample was the middle section of the first urina of the day. The urinary sample was placed in the liquid nitrogen immediately after sampling and stored at −86□ in a low-temperature refrigerator. The urinary 11-dehydrothromboxane B₂ (TXB₂) was determined by immunoenzyme assay using the reagent kit of Cayman Chemical company. All the experiments were completed in the Biological Institute of Tasly Research Academy. Information about patient grouping was double-blind for researchers and statisticians.

Results

The results were showed in table 2.

TABLE 2
Values of urinary 11-dehydrothromboxane B₂ in patients of each group
(ng/mmol) X ± SD

| Aspirin group | CDT group | Combination therapy group |
|---|---|---|
| (n = 50) | (n = 50) | (n = 50) |
| Urinary 11-dehydro | Urinary 11-dehydro | Urinary 11-dehydro |
| TXB₂ | TXB₂ | TXB₂ |
| (ng/mmol) | (ng/mmol) | (ng/mmol) |
| 25.05 ± 10.42 | 21.31 ± 8.14 | 20.53 ± 8.23 |

Conclusion: The excreted TXB2 between combination therapy group and aspirin group was significantly different in statistics (p<0.05). The excreted TXB2 between CDT group and aspirin group was also significantly different in statistics (p<0.05). The excreted TXB2 between combination therapy group and CDT group was not significantly different in statistics (p>0.05).

The experiment data demonstrated that CDT could reduce the excretion of urinary TXB2. That is to say, it could block the biosynthesis of thromboxane A₂ (TXA₂) in vivo and reduce platelet conglutination and coagulation. Accordingly, it could inhibit platelet aggregation and reduce the incidence of arteriosclerosis and myocardial infarction. The experiment results indicated that CDT had anti-aspirin resistance effect. CDT could improve life indexes in aspirin resistant patients with cardiovascular diseases.

According to the methods mentioned-above, tests were conducted on various preparations comprising RSM, such as danhong injection, qianglinaoxinkang, tongxinshu capsules, compound dangshen tablets, danxiang guanxin injection, danshen injection, jingzhi guanxin granules, jingzhi guanxin tablets, shuxintong capsules, xinnaoning capsules, guanxindanshao tablets, xinxinshu capsules, xiongxiangtongmai pills, guanxindanshen tablets, guanxindanshen drop pills etc. It was demonstrated that all the preparations comprising RSM had the effect of anti-aspirin resistance.

1. A method for treating anti-aspirin resistance in a subject, comprising administering at least one of RSM, RSM extract and a composition including at least one of RSM and RSM extract as an active ingredient to said subject in need of such treatment.

2. The method of claim 1 wherein said aspirin resistance is an aspirin resistant cardio-cerebrovascular disease.

3. The method of claim 2 wherein said aspirin resistant cardio-cerebrovascular disease includes at least one of coronary heart disease and angina pectoris.

4. The method of claim 1 wherein said composition includes a compound preparation including at least one of RSM and RSM extract.

5. The method of claim 4 wherein said compound preparation is selected from the group consisting of danhong injection, qianglinaoxinkang, tongxinshu capsules, compound dangshen tablets, danxiang guanxin injection, danshen injection, jingzhi guanxin granules, jingzhi guanxin tablets, shuxintong capsules, xinnaoning capsules, guanxindanshao tablets, xinxinshu capsules, xiongxiangtongmai pills, guanxindanshen tablets, guanxindanshen drop pills, compound danshen drop pills, and compound danshen tablets.

6. The method of claim 4 wherein said composition comprises RSM 30-180 parts, Radix Notoginseng 5-40 parts, Borneolum 0.3-2.5 parts, and adjuvants 10-40 parts.

7. The method of claim 6 wherein said composition comprises RSM 75-115 parts, Radix Notoginseng 14-20 parts, Borneolum 0.8-1.2 parts, and adjuvants 15-30 parts.

8. The method of claim 7 wherein said composition comprises RSM 90 parts, Radix Notoginseng 17.6 parts, Borneolum 1 part, and adjuvants 20 parts.

9. The method of claim 6 wherein said adjuvants include polyethylene glycol.

10. The method of claim 4 wherein said composition is selected from the group consisting of drop pills, spray solution, pellets, pills, granules, capsules, tablets, powder and oral liquid.
